(19) United States
(12) Patent Application Publication
(10) Pub. No.: US 2022/0225999 A1
(43) Pub. Date: Jul. 21, 2022

(54) Treatment of Unstable Plaque/Thrombus

(71) Applicant: MG Stroke Analytics Inc., Calgary (CA)

(72) Inventor: Mayank Goyal, Calgary (CA)

(21) Appl. No.: 17/609,775

(22) PCT Filed: May 11, 2020
(86) PCT No.: PCT/CA2020/050640

§ 371 Date: Nov. 8, 2021

Related U.S. Application Data
(60) Provisional application No. 62/846,467, filed on May 10, 2019.

(51) Int. Cl. A61B 17/12; A61L 24/04; A61M 25/09

(52) U.S. Cl. CPC A61B 17/12168; A61L 24/046; A61B 17/12136; A61B 2017/00004; A61B 17/12177; A61M 25/0905; A61B 17/12109

(57) ABSTRACT

Methods for the diagnosis and treatment of nonstenotic carotid plaques and symptomatic nonstenotic carotid disease (SyNC) are described. In particular, methods of evaluating the presence of unstable plaque/thrombus and methods of treatment that include deploying plaque stabilizers (PSs) into the cerebral vasculature are described. The invention further describes plaque stabilizers, uses of plaque stabilizers and plaque stabilizer kits.

### FIELD OF THE INVENTION

Methods for the diagnosis and treatment of nonstenotic carotid plaques and symptomatic nonstenotic carotid disease (SyNC) are described. In particular, methods of evaluating the presence of unstable plaque/thrombus and methods of treatment that include deploying plaque stabilizers (PSs) into the cerebral vasculature are described. The invention further describes plaque stabilizers, uses of plaque stabilizers and plaque stabilizer kits.

### BACKGROUND OF THE INVENTION

Acute ischemic stroke (AS) or Transient Ischemic Attack (TIA) are acute diseases where tissue death (infarction) may occur in the brain if timely treatment is not undertaken.

A common cause of AS/TIA is when an emboli breaks free from a development site (typically within the arterial system), which then travels into brain blood vessels. Emboli may have a variety of morphological and/or compositional characteristics, such as being predominantly fatty tissues (atherosclerosis plaque) and/or a blood clot (thrombus).

Embolic stroke of undetermined source (ESUS), often called cryptogenic stroke, is a diagnosis of exclusion that accounts for 9% to 25% of all ischemic strokes. The average recurrence rate of ESUS is 4.5% per year, despite prophylactic treatment with antithrombotic agents.

With improved cardiac imaging and prolonged monitoring for arrhythmia, it has been recognized that a substantial number of ESUS cases might actually be better classified elsewhere such as within the cardioembolic strokes caused by covert atrial fibrillation or as paradoxical emboli due to patent foramen ovale.

Clot composition of thrombectomy specimens shows a high platelet content in many cryptogenic patients which suggests that unrecognized arteriogenic causes are also common. According to the TOAST trial (Trial of ORG 10172 in Acute Stroke Treatment), the definition of large artery atherosclerosis strokes is based on the degree of stenosis where only patients with carotid or other extracranial/intracranial lesions >50% stenosis according to the NASCET (North American Symptomatic Carotid Endarterectomy Trial) criteria are included in this definition. However, numerous studies describe the association of certain plaque characteristics, such as intraplaque hemorrhage and plaque thickness with strokes in general and ESUS in particular, regardless of the degree of stenosis.

Various factors such as plaque surface irregularity on angiography is strongly associated with an increase in ipsilateral stroke risk irrespective of the stenosis degree. In addition, nonstenotic carotid plaques are much more common ipsilateral to the side of stroke in patients with ESUS. These findings suggest that nonstenotic carotid disease might play a role in stroke etiology (symptomatic nonstenotic carotid disease [SyNC]).

For reference, occurrence of unstable plaque/thrombus at the common carotid artery (CCA) bifurcation is discussed.

As is known, atherosclerosis plaques and/or thrombi may form in a number of locations in the body from a variety of triggering factors. One common sources of emboli causing AS/TIA is plaque and/or thrombus that forms at the common carotid artery (CCA) bifurcation where the CCA branches into the internal carotid artery (ICA) and the external carotid artery (ECA).

As atherosclerotic plaque grows within an artery, it will increasingly cause a narrowing or stenosis of the artery and hence a restriction to blood flow. As stenosis increases, a patient may become symptomatic as the decreased blood supply affects tissues distal to the obstruction. In addition, emboli may break off the plaque. Generally, symptoms caused by a narrowing of a vessel will not present until a vessel is more than 50% obstructed. In this case, if a patient becomes symptomatic due to stenosis (for example the patient experiencing sudden weakness) and is not showing symptoms of acute stroke (for example, loss of neurological functions), a number of treatment options are available as will be described below.

In situations where an emboli has broken free and the patient is showing signs of AS/TIA, the severity of symptoms, diagnosis of the location of the resting place of the emboli and/or the origin of the emboli may all contribute to a treatment option decision. For example, one common signal of a significant AS/TIA is amaurosis fugax which presents as a transient loss of vision in the ipsilateral eye. In this case, an emboli may have had origins within the common carotid artery (CCA) and specifically at the CCA bifurcation.

Importantly, there are also situations where stenosis of an artery such as the CCA, is less than 50% and the patient has or is exhibiting symptoms. Generally, in these cases, symptoms have presented not necessarily because of the blood flow restriction but due to emboli breaking free from the atherosclerotic plaque/thrombus which may then present various neurological symptoms.

These types of plaque/thrombus are referred to as unstable plaque/thrombus insomuch as they are characterized as plaque/thrombus where stenosis is less than 50% and where the patient is exhibiting symptoms.

For reference, FIG. 1 is a schematic diagram of a CCA bifurcation 100. The CCA bifurcation 100 includes a CCA 102, an ICA 104 and an ECA 106. A direction of blood flow 101 shows the normal direction of flow from the CCA 102 to both the ICA 104 and the ECA 106. Exemplary plaque deposits 108a, 108b and 108c are shown at locations where plaque could be deposited proximal to the CCA bifurcation 100. Plaque deposit 108a is located in the ICA 104 and extends annularly around the ICA. Plaque deposit 108b is located on a portion of the ECA 106. Plaque deposit 108c is located on a portion of the CCA 102. For the purposes of description, as an unstable plaque can be varying degrees of atherosclerotic tissue or thrombus and the proportions cannot be readily diagnosed or quantified, this description will refer to unstable plaque with the understanding that an unstable plaque may be comprised of varying proportions of atherosclerotic and thrombus material.

Furthermore, for the purposes of background description, it is important to note that blood supply to the brain is somewhat unique due in part due to the connection between ICAs on both sides of the body through the Circle of Willis. FIG. 2 is a schematic diagram of a Circle of Willis showing a left ICA and a right ICA, which are connected through two pathways: one comprising left and right anterior cerebral arteries and the anterior communicating artery, and the other comprising left and right posterior communicating arteries and left and right posterior cerebral arteries. As such, if blood flow is cut off to one CCA (the ipsilateral side), blood flow may still be maintained to the ipsilateral ICA through the Circle of Willis. The ECA also includes various cross connections where, in the event of occlusion of one ECA (e.g. at the CCA bifurcation; ipsilateral side), the cross connections can provide blood flow to the distal ipsilateral vessels. As is known, there are a number of anatomical variations between individuals that can provide a variety of cross connection patterns.

A variety of treatments are known for treating patients having various types and sizes of plaque at the CCA bifurcation and particularly those causing severe stenosis. For example, in the case of severe stenosis, one common procedure is carotid endarterectomy in which the plaque is removed surgically after opening the vessel. Another procedure is carotid stenting (also referred to as scaffolding) that involves placement of a metal stent (or scaffold) within the stenosed artery to open the vessel and provide a means of holding the plaque against the arterial wall. One particular advantage of using metal stents is that metal stents are radio-opaque which facilitates deployment procedures as they are visible with imaging equipment.

Importantly, in cases where carotid stenting is performed using a metal stent, the physician must consider the short-term and long-term risks and benefits of deploying a metal stent to treat the particular plaque/thrombus characteristics. One important consideration is that once a metal stent has been deployed, it cannot be removed; hence future treatment options are thereafter reduced when a metal stent has been used. Permanent placement of a metal carotid stent can provide positive benefits of opening a vessel and thus improving blood flow whilst reducing the risk of the plaque breaking free, but it can also result in long-term complications such as in-stent stenosis. If a longer-term complication does arise, there are then fewer options available.

In general, when a patient has exhibited symptoms, the degree of stenosis of the vessel due to plaque plays a major role in decision making regarding intervention (surgery or stenting). In addition, presence of symptoms related to the plaque/thrombus are important as well. This approach is backed by several randomized controlled trials. For example, for symptomatic patients with >70% stenosis, carotid endarterectomy has shown clear benefit.

There is also increasing data for intervention in symptomatic patients with 50-69% stenosis as well.

For asymptomatic patients with severe stenosis, there is quite a bit of variation of practice around the world as the data is equivocal. In such situations, other factors may come into play such as patency of the Circle of Willis, patient wishes, surgeon/interventionist perceived procedural risk amongst other factors.

As noted above, when a patient has exhibited symptoms, and upon diagnosis, the plaque/thrombus shows relatively low stenosis (<50%), the plaque may also have an unstable appearance where a physician may consider that the risk of the plaque/thrombus breaking free within a relatively short time frame is reasonably high.

There are a number of techniques that help diagnose unstable plaque. It has been shown that plaques may get inflamed and become unstable (for example, such plaques may show enhancement of high-resolution contrast enhanced MR imaging). Hemorrhage into the plaque may also lead to unstable plaque.

In summary, the features of non-stenotic plaques (unstable plaques) that are associated with higher risk of stroke and imaging modalities used to identify them are shown in Table 1.

TABLE 1
Features of Non-Stenotic Plaques That Are
Associated With Higher Risk Of Stroke

| Plaque Feature | Imaging Modality |
|---|---|
| Ulceration | MRI, CTA |
| Intraplaque Hemorrhage | MRI |
| Fibrous Cap Rupture | MRI |
| Plaque Thickness | MRI, CTA |
| Plaque Echolucency | Ultrasound |
| Lipid-Rich Core | MRI |
| Surface irregularity | MRI, CTA |
| Carotid Web | CTA |
| Changing morphology on | CTA, MRA |
| short term follow up | |

CTA-Computed Tomography Angiography
MRI-Magnetic Resonance Imaging

Optical Coherence Tomography (OCT) is another imaging technique that could be used to ascertain features relevant to unstable plaque.

Accordingly, there has been a need for improved treatment options for unstable plaques that in particular may provide a solution to stabilize the plaque whilst maintaining the potential for a surgeon to conduct future treatments.

### SUMMARY OF THE INVENTION

The invention describes medical procedures, and in particular to methods for treatment of nonstenotic carotid plaques and symptomatic nonstenotic carotid disease (SyNC). The invention further describes plaque stabilizers (PSs), uses of PSs and PS kits for the treatment of unstable plaque and/or thrombus.

In a first aspect, the invention provides a method for treatment of an unstable plaque/web/thrombus at a zone of interest in a patient with or without significant stenosis, comprising the step of: deploying a plaque stabilizer (PS) over the unstable plaque/web/thrombus to prevent further embolization of plaque/thrombus fragments and to stabilize the unstable plaque for a therapeutically effective time period.

In one embodiment, prior to the step of deploying, the method includes the step conducting an imaging analysis of the zone of interest via at least one imaging modality, the imaging modalities including any one of or a combination of computed tomography angiography (CTA), magnetic resonance imaging (MRI), ultrasound or optical coherence tomography (OCT) to determine morphological characteristics of a plaque at the zone of interest.

In another embodiment, the step of conducting an imaging analysis includes a determination of any one or more of plaque ulceration, intraplaque hemorrhage, fibrous cap rupture, plaque thickness, plaque echolucency lipid-rich core, surface irregularity, carotid web and changing morphology on short term follow-up.

In another embodiment, the step of conducting an imaging analysis includes the step of applying a score representing the existence or not of any one of or a combination of plaque ulceration, intraplaque hemorrhage, fibrous cap rupture, plaque echolucency, lipid-rich, surface irregularity, carotid web and changing morphology on short term follow-up core and/or a measurement of plaque thickness when enabled by the imaging modality.

In other aspects, the invention contemplates combinations of the following:
The PS is resorbable that is resorbable within the body over a resorb time.
The PS is non-resorbable.
The zone of interest is at or adjacent to a bifurcation of a Common Carotid Artery (CCA) into an Internal Carotid Artery (ICA).
The step of deploying includes the step of substantially arresting blood flow adjacent to the unstable plaque prior to deploying the PS.
The step of substantially arresting blood flow adjacent to the unstable plaque comprises advancing a balloon guide catheter (BGC) into the CCA proximal to the unstable plaque and inflating a first balloon to occlude blood flow through the CCA.
The step of substantially arresting blood flow at the unstable plaque further comprises advancing a micro-balloon (MB) through the BGC and inflating the MB in the ECA adjacent the CCA bifurcation.
The method includes a step of checking to determine if blood has been arrested at the unstable plaque before the step of deploying the PS.
The method includes the step of establishing retrograde flow through the BGC to remove debris adjacent the CCA bifurcation.
The PS is self-expanding and has a pore size enabling the PS to act as a distal protection device (DPD) during PS deployment.
The resorb time is one week or less.
The resorb time is one month or less.
The resorb time is two months or less.
The PS is a drug-eluting resorbable PS.
The drug-eluting resorbable PS is adapted to release one or more anti-mitotic drugs and/or one or more anti-thrombogenic drugs and/or one or more anti-inflammatory drugs.
The anti-inflammatory drugs comprise heparin.
The resorbable PS is adapted for reduced thrombogenicity.
The PS has a pore size sufficiently small to prevent pieces of the unstable plaque from passing through the PS and breaking free.
The PS has a taper to accommodate for the reduction of diameter between the CCA and ICA.
The PS is a self-expanding resorbable PS.
The resorbable PS no longer exists at the site of the unstable plaque.

In another aspect, the invention relates to the use of a resorbable PS to stabilize an unstable plaque for a therapeutically effective time period in a patient at or adjacent to a bifurcation of a Common Carotid Artery (CCA) into an Internal Carotid Artery (ICA) and an External Carotid Artery (ECA) (the CCA bifurcation) in a patient. The resorbable PS may be deployed under substantial arrest of blood flow adjacent to the unstable plaque.

In another aspect the invention provides a kit for the treatment of an unstable plaque at or adjacent to a bifurcation of a common carotid artery (CCA) to an internal carotid artery (ICA) and external carotid artery (ECA) in a patient, the kit comprising: at least one guide catheter (GC) configured for placement of the GC proximal to the unstable plaque; at least one plaque stabilizer deployment device (PSDD) configured for telescopic engagement within the GC and for placement distal to the unstable plaque; at least one guide wire (GW) configured for telescopic engagement within the MC and for placement distal to the unstable plaque; and at least one PS configured to the PSDD for placement adjacent to the unstable plaque and deployable through from the PSDD.

The PS may be resorbable over a resorb time.

The kit may include at least one balloon guide catheter (BGC) for occluding blood flow through the CCA and/or at least one micro-balloon (MB) for occluding blood flow through the ECA and/or at least two resorbable PS assemblies each having a resorbable PS, and where the resorbable PSs have at least one different structural and/or functional property from each other, selected from any one of or a combination of PS diameter, PS length, PS taper, PS compressive stiffness, PS pore size; PS drug coating and PS resorb time.

In another aspect, the invention provides a plaque stabilizer (PS) for deployment over an unstable plaque/web/thrombus comprising: a cylindrical body having a plurality of pore openings in the range of 110-250 microns diameter and a void space of greater than 50% of the cylindrical body, the cylindrical body collapsible within a microcatheter and deployable from the microcatheter for placement over the unstable plaque/web/thrombus and wherein the cylindrical body is self-expanding upon deployment within an artery.

In other aspects, the invention contemplates combinations of the following:
The PS is comprised of a resorbable material having a resorb time of one week or less.
The PS is comprised of a resorbable material having a resorb time of one month or less.
The PS is comprised of a resorbable material having a resorb time of two months or less.
The PS is poly lactic-co-glycolic acid.
The PS is a cylindrical body having a weave of poly lactic-co-glycolic acid filaments, the filaments having a diameter in the range of 30-50 microns.
The PS has at least two zones, a first distal zone having pore opening in the range of 110-250 microns diameter and second proximal zone having pore openings greater than 250 microns.
The cylindrical body has an overall length of 3-5 cm.
The first distal zone is 70-80% of the overall length of the cylindrical body.
The second proximal zone is 20-30% of the overall length of the cylindrical body.
The PS is metal.

### BRIEF DESCRIPTION OF THE DRAWINGS

Various objects, features and advantages of the invention will be apparent from the following description of particular embodiments of the invention, as illustrated in the accompanying drawings. Similar reference numerals indicate similar components:

FIG. 1 is a schematic diagram of a CCA bifurcation.

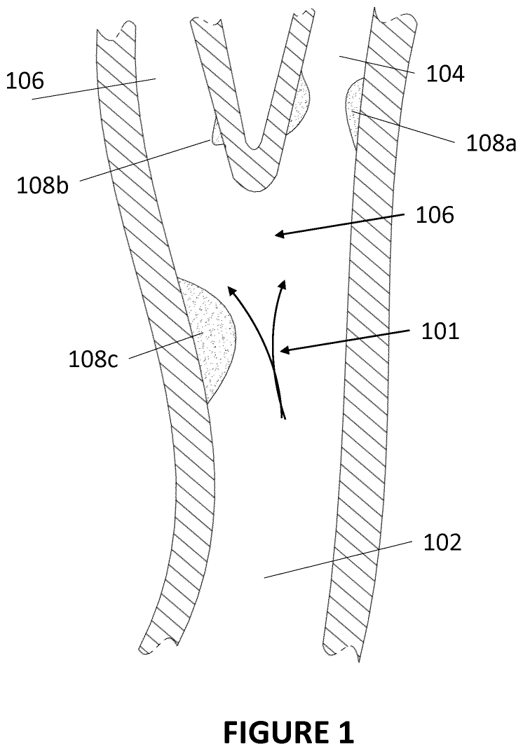

FIG. 2 is a schematic diagram of the anatomy of a typical Circle of Willis.

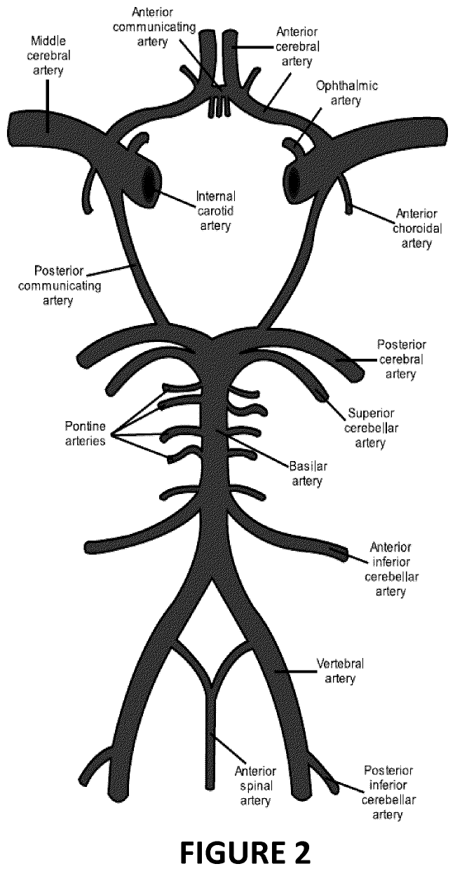

FIG. 3 is a flow chart of a method for treatment of an unstable plaque, according to one embodiment of the invention.

FIG. 4 is a schematic diagram of a CCA bifurcation showing an unstable plaque and a balloon guided catheter (BGC) inserted in a CCA with a first balloon being inflated, according to one embodiment.

FIG. 5 is a schematic diagram of the CCA bifurcation of FIG. 4, with the BGC extending into the ECA, the first balloon being fully inflated and a second balloon being inflated.

FIG. 6 is a schematic diagram of the CCA bifurcation of FIG. 5, with the second balloon fully inflated and a guide wire inserted through an aperture of the BGC and into the ICA, past the unstable plaque.

FIG. 6A is a schematic diagram showing a combined balloon guide catheter (BGC) and micro-balloon (MB).

FIG. 7 is a schematic diagram of the CCA bifurcation of FIG. 6, showing a plaque stabilizer deployment device (PSDD) extending along the guide wire.

FIG. 7A is a schematic diagram showing various details of one embodiment of a PSDD.

FIG. 8 is a schematic diagram of the CCA bifurcation of FIG. 7, with the guide wire removed.

FIG. 9 is a schematic diagram of the CCA bifurcation of FIG. 8, showing a plaque stabilizer (PS) assembly that has been advanced inside the PSDD.

FIG. 10 is a detailed view of a portion of a proximal end of a PS assembly as shown in FIG. 9.

FIG. 11 is a schematic diagram of the CCA bifurcation of FIG. 9, showing a resorbable PS of the PS assembly being deployed and acting as a distal protection device.

FIG. 11A is a schematic diagram showing a resorbable PS being deployed over an unstable plaque.

FIG. 12 is a schematic diagram of the CCA bifurcation of FIG. 11, showing the resorbable PS being further deployed.

FIG. 13 is a schematic diagram of the CCA bifurcation of FIG. 12, showing the resorbable PS in the deployed position with the BGC and the PSDD having been removed.

FIG. 14 is a schematic diagram of a resorbable PS being deployed without flow cessation.

FIG. 15 is a schematic diagram of a PS in accordance with one embodiment of the invention.

### DETAILED DESCRIPTION OF THE INVENTION

### Introduction and Rationale

The inventor understood that a significant number of strokes are categorized as being from unknown sources referred to herein as embolic strokes of underdetermined source (ESUS).

ESUS may be caused by a number of different etiologies that are currently unrecognized or uncategorized with current investigations. For example, some ESUS might be better categorized as cardioembolic strokes due to covert atrial fibrillation whereas other ESUS might be better categorized as symptomatic nonstentotic carotid disease (SyNC). SyNC may be related to various plaque features including ulceration, intraplaque hemorrhage, fibrous cap rupture, plaque thickness, plaque echolucency, lipid-rich core, surface irregularity, carotid web and changing morphology on short term follow-up which can be qualitatively and quantitatively assessed as “unstable plaque”.

An unstable plaque will typically have produced symptoms in the ipsilateral circulation (e.g. amaurosis fugax, TIA) and have an irregular shape and generally be adhered to a smaller proportion of the arterial vessel as compared to an atherosclerotic plaque where the degree of stenosis is greater than 50%. Due to its irregular shape, blood flow around the unstable plaque may be turbulent which may lead to the plaque, or portions of the plaque, breaking free.

The diagnosis of unstable plaque may be made using a combination of factors after a patient has exhibited various symptoms. These factors include: presence of irregular plaque at the ipsilateral carotid origin determined by imaging including ulceration, intraplaque hemorrhage, fibrous cap rupture, plaque thickness, plaque echolucency, lipid-rich core, surface irregularity, carotid web and changing morphology on short term follow-up there; absence of any other risk factors (e.g. cardiac issues such as atrial fibrillation); strokes limited to that circulation on diffusion MRI; presence of blood products within the plaque or enhancement of the plaque on high resolution MRI; and presence of ‘donut sign’ on CT angiography.

Modification in the shape or morphology of the plaque over short term repeat imaging is another pointer.

Current literature does not advocate procedures to acutely manage these plaques to immediately reduce the risk of sudden embolic stroke without potentially introducing long term risks. Further, it is not uncommon for an unstable plaque to stabilize or settle down by itself over the next several weeks. Therefore, patients with unstable plaques may be managed with heparin and other anti-coagulation drugs in hopes that the unstable plaque with stabilize before it embolizes into the distal circulation.

The present inventor, having a background in the medical treatment of strokes and TIAs, is familiar with technological developments occurring in this field in recent years. The inventor recognized that further options must be developed for the acute treatment of AS/TIAs that do not introduce long term health risks. The inventor realized that it is desirable to stabilize an unstable plaque in the short term to minimize the risk of it suddenly breaking free without introducing further or long-term risks.

### Terminology

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Spatially relative terms, such as “distal”, “proximal”, “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a feature in the figures is inverted, elements described as “under” or “beneath” other elements or features would then be oriented “over” the other elements or features. Thus, the exemplary term “under” can encompass both an orientation of over and under. A feature may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present.

It will be understood that, although the terms “first”, “second”, etc. may be used herein to describe various elements, components, etc., these elements, components, etc. should not be limited by these terms. These terms are only used to distinguish one element, component, etc. from another element, component. Thus, a “first” element, or component discussed herein could also be termed a “second” element or component without departing from the teachings of the present invention. In addition, the sequence of operations (or steps) is not limited to the order presented in the claims or figures unless specifically indicated otherwise.

In this description, plaque stabilizers (PSs) and resorbable PSs are described. PSs as described herein are different to the stents utilized in other vascular procedures primarily to the extent that a stent is utilized for angioplasty and hence, has inherent properties intended to open or expand a narrowed vessel. In contrast, a PS has the primary function of covering a plaque for the purpose of stabilizing its outer surfaces without inherent angioplasty properties.

Other than described herein, or unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Unless otherwise defined, all technical and scientific terms used herein have the same meaning as commonly understood by one of ordinary skill in the art to which this invention belongs.

Various aspects of the invention will now be described with reference to the figures. The invention may, however, be embodied in many different forms and should not be construed as limited to the embodiments set forth herein; rather, these embodiments are provided so that this disclosure will be thorough and complete, and will fully convey the scope of the invention to those skilled in the art. Moreover, the drawings are not necessarily drawn to scale and are intended to emphasize principles of operation rather than precise dimensions.

### Unstable Plaque Diagnosis

Unstable plaque at the ICA/ACA bifurcation may be qualitatively and quantitatively assessed using a variety of imaging techniques as listed in Table 1. Depending on the imaging techniques available, the diagnosing physician can determine whether an unstable plaque is present based on an objective determination of the existence of various features of the plaque. That is, an objective score can be applied to the plaque to determine whether it should be characterized as unstable or not. As shown in Table 2, for features such as ulceration, intraplaque hemorrhage, fibrous cap rupture, plaque echolucency and lipid-rich core, the existence of any of these features will provide a + score. For plaque thickness, an objective measurement of the plaque thickness above a threshold value (for example 3 mm) is a yes or no determination.

TABLE 2
Factors and Scoring for Objective
Determination of Unstable Plaque

| Plaque Feature | Imaging Modality | Score |
|---|---|---|
| Ulceration | MRI, CTA | + − |
| Intraplaque Hemorrhage | MRI | + − |
| Fibrous Cap Rupture | MRI | + − |
| Plaque Thickness | MRI, CTA | >3mm y/n |
| Plaque Echolucency | Ultrasound | + − |
| Lipid-Rich Core | MRI | + − |
| surface irregularity | MRI, CTA | + − |
| carotid web | CTA | + − |
| changing morphology on | CTA, MRI | + − |
| short term follow-up | | |

The diagnosing physician may or may not have all imaging modalities available to make an unstable plaque determination. Hence, for example in the event that only CTA is available a + score for ulceration in combination with a plaque thickness above the threshold would suggest unstable plaque. Similarly, if MRI is available a + score for any one or more of ulceration, intraplaque hemorrhage, fibrous cap rupture or lipid-rich core together with a plaque thickness above the threshold would suggest unstable plaque. Minus − values would not be suggestive of unstable plaque.

While the determination of unstable plaque will not automatically result in a decision to treat the unstable plaque through intervention, as many other factors can come into play including for example the age of the patient and their past history, if those other factors are suggestive of a positive outcome, a final decision to treat may be made. Importantly, the step of objectively determining if an unstable plaque exists can be used as a step in the overall treatment process.

### Systems and Methods for Treatment of Unstable Plaque

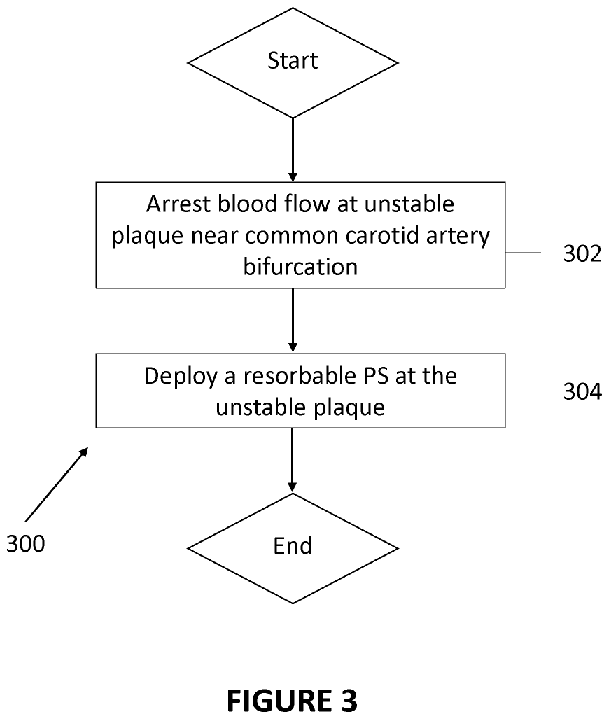

An example method for the treatment of an unstable plaque at the CCA bifurcation after an unstable plaque has been identified will now be described with reference to FIGS. 3 to 13. In this description, a plaque stabilizer (PS) may be non-resorbable or resorbable. Non-resorbable and resorbable PSs have the same general properties with the primary difference being the stability of the PS in the body over periods of time. The following discussion is made with reference to a resorbable PS having the following properties:
a. resorbable over a period of time (for example 1 week to a few months);
b. self-expanding upon deployment from a catheter;
c. outward spring strength sufficient to engage against an arterial wall,
d. low porosity relative to the size of potential emboli breaking off the surface of the unstable plaque whilst enabling blood cells to pass through the PS; a typical pore size may be 110-250 microns;
   and optionally may be:
e. tapering to enable effective placement in tapered arterial vessels and/or
f. a substrate for local drug delivery or to reduce thrombogenicity.

In the context of the above functionality, “self-expanding” generally means that the PS can be compressed and slidingly engaged within a smaller catheter. Upon emergence of the PS from the catheter, the PS will expand under its inherent spring pressure contained with the matrix of wires/filaments of the PS and is sized to expand to the size of the vessel it is being deployed in. “Outward spring strength” generally means that the inherent spring pressure contained with the matrix of wires/filaments of the PS is sufficient to expand to the size of the vessel it is being deployed in generally without a sufficient force to open the vessel.

FIG. 3 shows a flow chart of a method 300 for treatment of an unstable plaque, according to one embodiment. The method includes, at step 302, substantially arresting blood flow adjacent to the CCA bifurcation and the unstable plaque and at step 304, deploying a resorbable PS over the unstable plaque to stabilize the unstable plaque for a therapeutically effective time period and wherein the PS is resorbed over a resorb time.

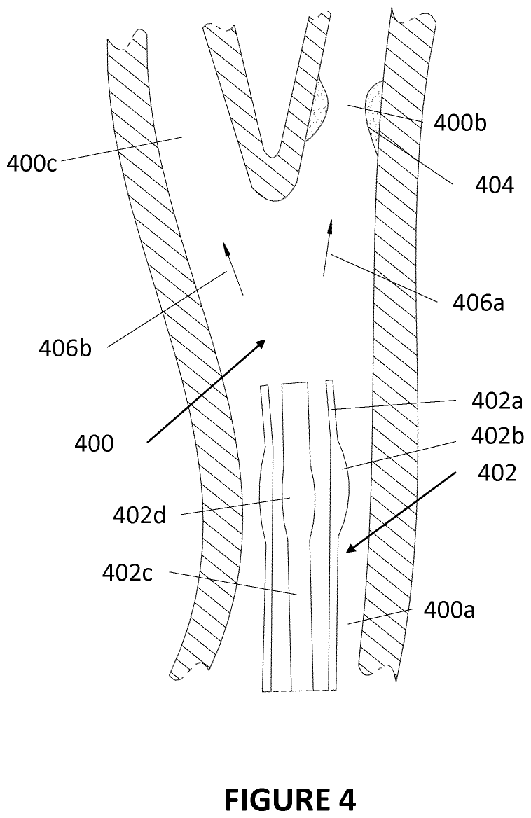

The method 300 will be further illustrated with regard to the example steps shown in FIGS. 4 to 13. FIGS. 4 to 13 show similar features. Features that are common between FIGS. 4 to 13 have not necessarily been relabeled for clarity of the drawings.

FIG. 4 is a schematic of a CCA bifurcation 400 having a CCA 400a, an ICA 400b and an ECA 400c. FIG. 4 also shows an unstable plaque 404 located in the ICA 400b, and a balloon guide catheter (BGC) 402 inserted into the CCA 400a and proximal to the CCA bifurcation.

Flow lines 406a, 406b show the direction of blood flow from the CCA 400a to both the ICA 400b and the ECA 400c.

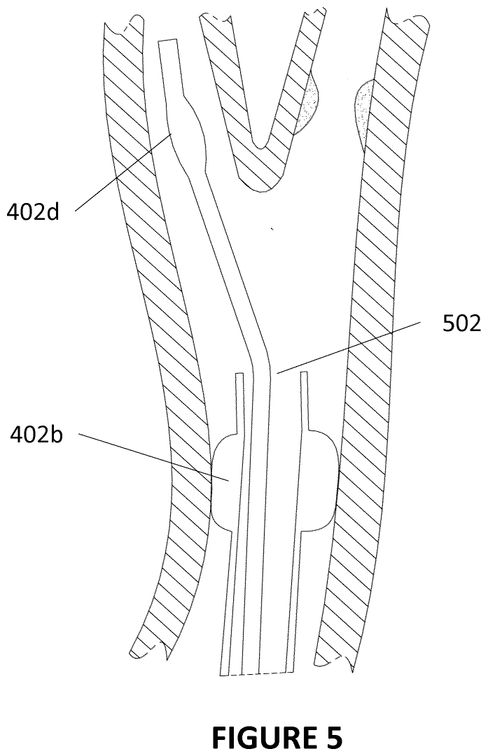

The balloon guide catheter (BGC) 402 includes a first catheter 402a having a balloon 402b. In FIG. 4, the balloon 402b is in the process of being inflated and FIG. 5 shows the balloon fully inflated.

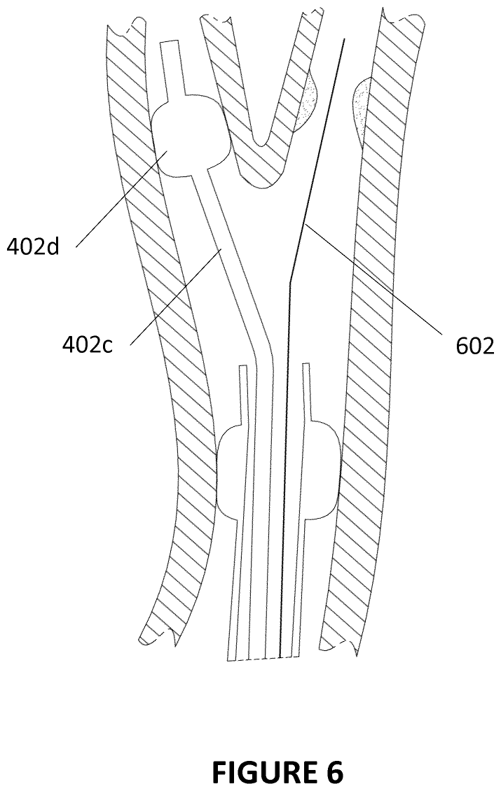

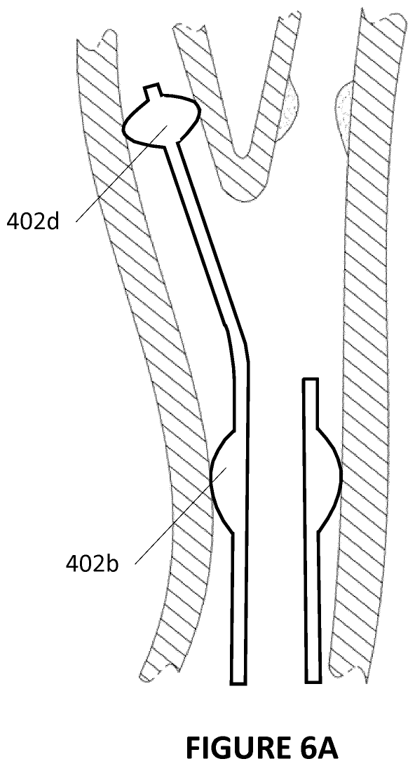

Within the BGC is a micro-balloon (MB) 402d (forming part of a microcatheter 402c such that it can be inserted through the BGC and still leave suitable space for a resorbable PS to be deployed through the BGC). As shown in FIG. 4, the MB is advanced through the first BGC in an uninflated configuration. The MB is expandable to a caliber to completely fill the lumen of the ECA and be occlusive as shown in FIG. 6.

In an alternative design as shown in FIG. 6A, the BGC and MB are constructed as one piece where the MB is attached to the tip of the BGC and both the balloons share a common connection for inflation from the outside. The distance between the tip of the BGC and the distal micro-balloon would typically be 5-10 cm. The purpose of this alternate design is to have greater space within the lumen of the BGC 402b to accommodate the resorbable PS.

The BGC 402 (and MB if a unitary design) may be inserted into the CCA 400a by known techniques. For example, the BGC 402 may be inserted through the aortic arch according to standard procedures. The BGC 402 is then manipulated to be in the CCA 400a proximal to the unstable plaque 404, and the balloon on the BGC 402b is inflated as described above.

Once inflated, the first balloon 402b arrests antegrade flow through the CCA, ICA and ECA.

Turning now to FIG. 5 and FIG. 6, as shown the MB 402d is in a position to be fully inflated and the second catheter 402c of the MB 402d has been advanced through an aperture 502 of the BGC 402a and into the ECA 400c. The MB 402d is in the process of being inflated in FIG. 5. When inflated, the two balloons (BGC and MB) prevent essentially all antegrade flow from the CCA and retrograde flow down the ECA thus providing a substantially zero flow area at the level of the unstable plaque to conduct a stenting procedure.

While flow in the CCA, ICA and ECA on the ipsilateral side has been stopped, flow through the Circle of Willis (COW) and other vessels will usually provide enough circulation to keep the brain alive for a period of time. Moreover, as is understood, there are variations in patients' anatomies that may affect how a surgeon chooses to conduct a procedure having consideration to the specifics of a case. However, generally it is desirable that all procedures be conducted as quickly as possible to minimize the time where blood flow through the ipsilateral CCA is being occluded.

Importantly, the aperture 502 of the BGC allows selective communication between the BGC and the treatment area.

### Deployment Procedures

An example deployment procedure is conducted with reference to FIGS. 6-13. FIG. 6 is a schematic diagram of the CCA bifurcation 400, with the MB 402d fully inflated. As noted above, with both the balloons 402b, 402d fully inflated, blood flow adjacent the unstable plaque has been substantially arrested.

With blood flow arrested, a guide wire or microwire 602 (hereinafter referred to as a “guide wire”, for simplicity) is extended though the BGC 402a, through the aperture 502 and into the ICA 400b, past the unstable plaque 404. The guide wire 602 is placed to enable the deployment of a resorbable PS over the plaque as described below.

In various embodiments, the guidewire may have a distal protection device (DPD), such as a basket with small pores that allow blood to go through but would capture any emboli dislodged during the procedure (not shown) to provide an additional level of protection against procedural strokes. However, as explained below the need for a DPD is reduced by the PSs described herein.

Generally, when access to the desired position has been achieved by advancing a GW to the unstable plaque, the PSDD 702 is then advanced over the guide wire 602 to the desired position.

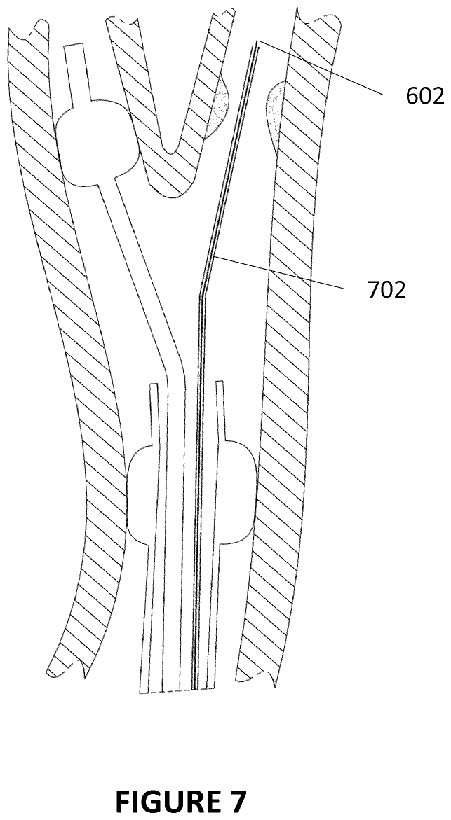

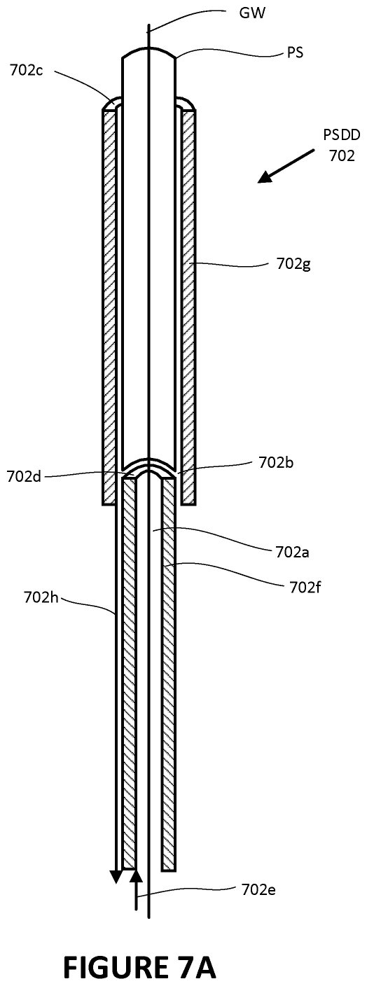

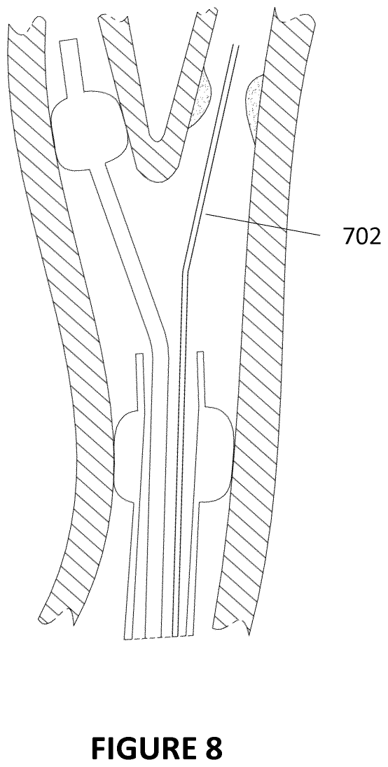

With the guide wire in place, FIG. 7 shows a plaque stabilizer deployment device (PSDD) 702 extending over the guide wire 602 to a position distal to the unstable plaque 404. Depending on the particular equipment utilized, the guide wire may be removed (FIG. 8) before deployment or after.

FIG. 7A shows a representative system for deployment of a PS.

As shown, the PSDD 702 has an inner lumen 702a and an outer lumen 702b. The inner lumen is defined by an inner sheath 702f and allows the passage of a guide wire GW. The outer lumen is defined by an outer sheath 702g and operatively retains the inner sheath. The inner sheath extends proximally at least a distance to enable the outer lumen to be withdrawn over it during deployment as explained below.

In general operation, the inner sheath can be held at a desired position within the vasculature by holding a distal end 702e of the inner sheath where the arrow 702e represents a holding force. The outer lumen retains the PS within the outer sheath adjacent a distal end 702c of the outer sheath. The outer sheath may be drawn proximally relative to the inner sheath as shown by arrow 702h. As the PS abuts against a distal end 702d of the inner sheath, proximal movement of the outer sheath relative to the inner sheath will cause the PS to emerge and expand from the distal tip 702c of the outer sheath.

After the guide wire is then removed through the PS and the entire system (without the PS) can be withdrawn from the body.

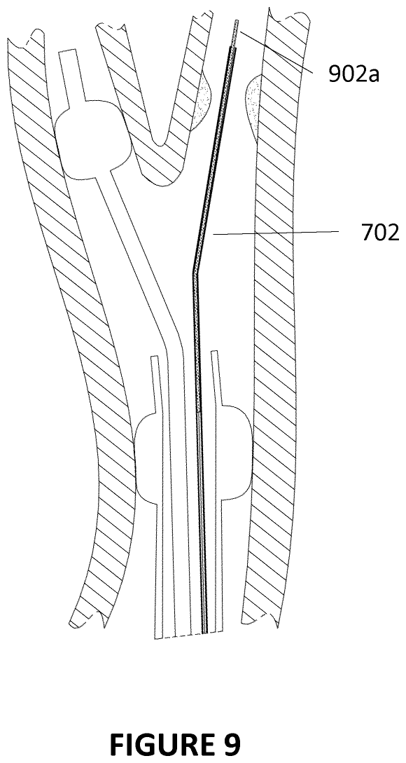

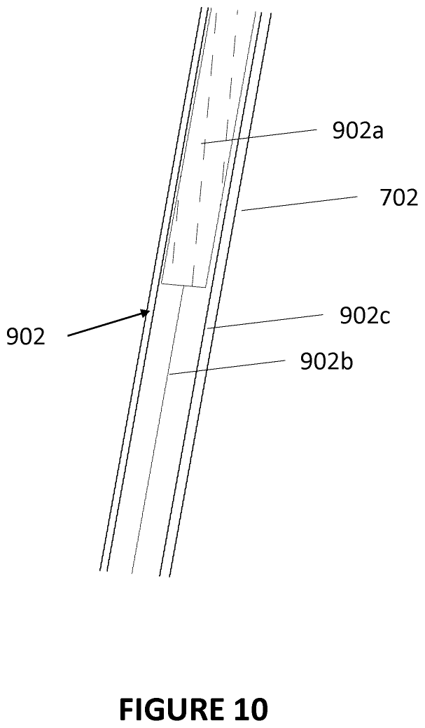

FIGS. 9 and 10 show another example of deployment. In this example, the outer sheath 902c (containing the resorbable PS 902a) has been advanced the desired position and the guide wire 602 has been removed. An engagement or push wire 902b connected to or engageable with the resorbable PS and is used to hold the resorbable PS 902a in position while the outer sheath 902c is removed in the proximal direction.

FIG. 9 also shows the resorbable PS 902a extending slightly beyond the outer sheath 902c.

The resorbable PS 902a may include certain features complementary with its deployment at the unstable plaque 404. For example, the resorbable PS 902a may be made of poly (lactic-co-glycolic) acid (PLGA) or any other material that is sufficiently rigid but may dissolve in the blood stream without deleterious effects. In an embodiment, the resorbable PS 902a may be adapted for reduced thrombogenicity. Certain features of such PSs can include PSs with specific coatings or geometries. In one embodiment, the resorbable PS 902a has a pore size sufficiently small to prevent small pieces of the plaque emerging through the pores and breaking free whilst providing sufficient outward force to maintain and outward pressure against the plaque and the adjacent arterial walls.

In an embodiment, although not required, the resorbable PS 902a may be a drug-eluting resorbable PS. For example, the drug-eluting resorbable PS may be adapted to release one or more anti-mitotic drugs and/or one or more anti-thrombogenic drugs and/or one or more anti-inflammatory drugs. The anti-inflammatory drugs may include heparin or warfarin, or a combination thereof, which may help stabilize the plaque.

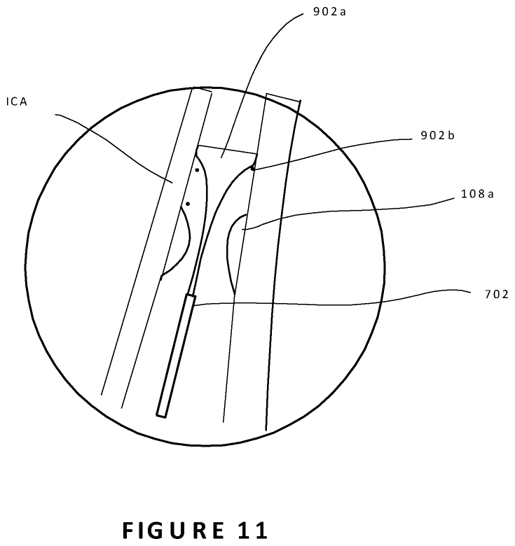

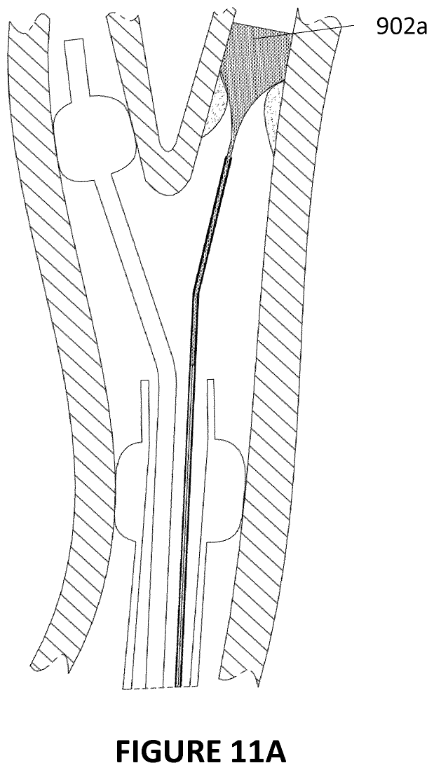

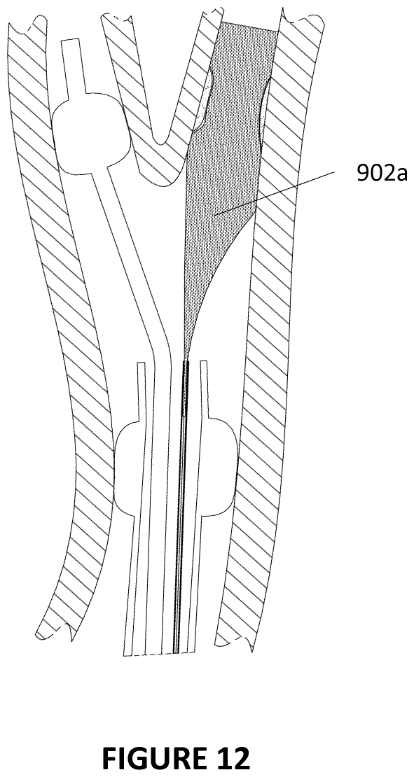

FIGS. 11, 11A and 12 shows the resorbable PS 902a being deployed. Specifically, the sheath 902c is withdrawn while the resorbable PS 902a is held in position by the engagement or push wire 902b. As the resorbable PS 902a expands it pushes against and/or compresses the unstable plaque 108a, thereby stabilizing the unstable plaque. Once the resorbable PS 902a is fully unsheathed, the engagement/push wire is withdrawn together with the PSDD.

The resorbable PS 902a may then remain at the site for a therapeutically effective time period and/or until it is resorbed. During the therapeutically effective time period the unstable plaque 404 may convert to atherosclerotic plaque, may dissolve in the blood stream and/or may be absorbed by the blood vessel of the ICA 400b, or a combination thereof. In an embodiment, the therapeutically effective time period and/or resorb time period may be less than one week. In another embodiment, the therapeutically effective time period and/or resorb time period may be less than one month, less than two months or less than three months. The length of the therapeutically effective time period and/or resorb time period may be determined by a number of factors including: how unstable the plaque is; the desired treatment outcome; the type of PS that is deployed; and the postoperative treatment protocol. After the therapeutically effective time period, the resorbable PS 902a may have substantially resorbed into the blood stream.

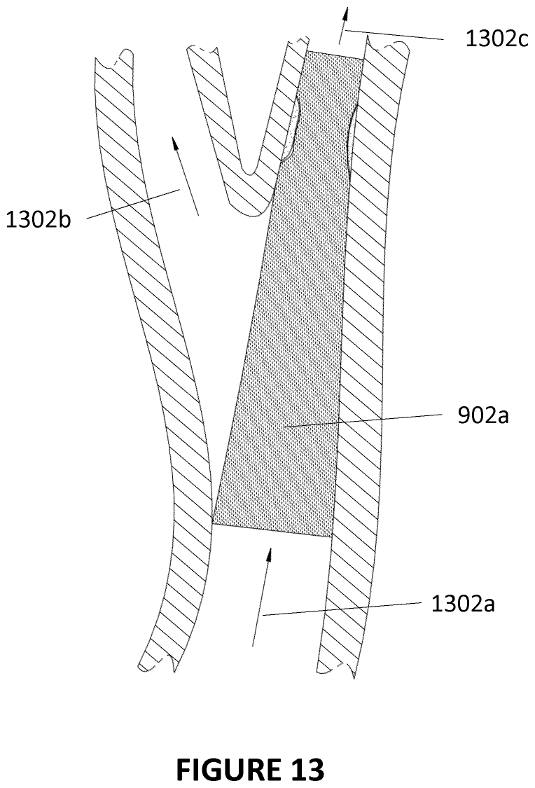

FIGS. 12 and 13 show the resorbable PS 902a deployed or bearing against the unstable plaque. The diameter, circumference and length of the resorbable PS 902a is merely exemplary. For example, the resorbable PS 902a may extend into the ECA 400c, depending on the geometry of the resorbable PS.

Generally, during and/or after the resorbable PS 902a deployment, debris is removed from the area via suction through the BGC 402. In another embodiment, a filter may be used to remove any accumulated debris.

Once the resorbable PS 902a is deployed, the PSDD is removed, the first balloon 402b and the MB 402d are deflated and removed, thus re-establishing flow. Blood flow lines 1302a,1302b,1302c show that normal blood flow from the CCA 400a to both the ICA 400b and the ECA 400c has been restored. As shown by the flow lines 1302a,1302c, blood may pass within the deployed resorbable PS 902a.

In the embodiment shown in FIG. 13, the resorbable PS 902a partially occludes the ECA 400c. Specifically, while the resorbable PS extends into the CCA 400a, at least some blood may be able to flow around or over the edges of the resorbable PS 902a and arterial walls and/or through pores in the resorbable PS. In another embodiment, the resorbable PS 902a may completely cover the origin of the ECA 400c, however, blood flow to the ECA is still maintained by virtue of the Circle of Willis and other cross-connections, described above. The proximal end of the resorbable PS may also be provided with a larger pore opening at this region of the PS.

Before and during the procedure, an anti-platelet and anti-coagulation drug regime may help reduce the risk that any debris released during the procedure will form a clot.

The procedure (from insertion of the BGC/MB and PS placement to removal), may be accomplished within about 3-5 minutes.

Importantly, the procedure utilizing a resorbable PS does not affect the ability to do other procedures in the future in the event of stenosis, growth or changes to the plaque at the site and/or a continued unstable appearance of the plaque. That is, to the extent that the PS has dissolved and the plaque has characteristics that may warrant the same or different treatment, these future procedures may be conducted.

### Alternate Techniques

### Alternate 1

In another embodiment, the resorbable PS is deployed without complete flow cessation by the BGC and/or MB. In a first alternate technique, the BGC is positioned as described above and a guidewire and PSDD are advanced past the unstable plaque utilizing the techniques described above.

Preferably, during the advancement of the GW and PSDD to beyond the clot, the balloon on the BGC is inflated and active aspiration is conducted during this step to produce transient retrograde flow thus reducing the chance of distal emboli.

The PS assembly is advanced over the guide wire and deployed.

The guide wire is withdrawn through the PS, the BGC is deflated and all equipment is withdrawn.

### Alternate 2

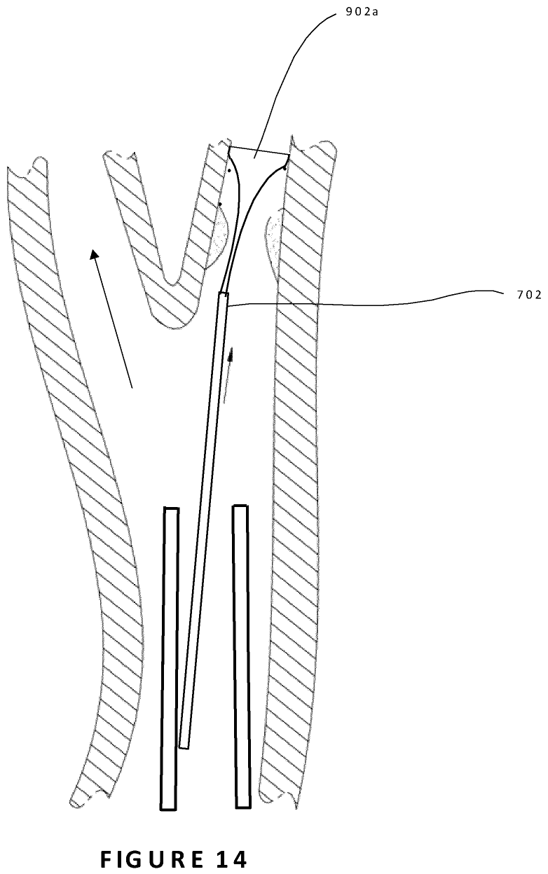

In a second alternate technique, the procedure is conducted without any balloons and hence without flow cessation as shown in FIG. 14. This technique provides an advantage over single or double balloon techniques by reducing the potential for blood pressure fluctuations during the procedure. That is, during a balloon technique, the cessation of blood flow can stimulate the carotid body (carotid glomus) at or adjacent to the CCA bifurcation which can cause significant blood pressure fluctuations during the procedure. As a result of this effect, single or double balloon procedures are generally conducted with an anesthetist to control patient blood pressure as necessary.

Accordingly, procedures conducted without the need of an anesthetist are generally advantaged by speed and cost.

Importantly, if the resorbable PS deployment is conducted without flow cessation, the resorbable PS can act as distal protection device (DPD) as explained below.

### Distal Protection Devices

As introduced above, current metal stenting procedures of stenosed vessels will usually deploy a distal protection device (DPD) mounted on the guide wire prior to stent deployment. A DPD is typically an inverted basket that can be advanced in a collapsed state past the plaque and deployed by withdrawing a protective sheath. After the DPD is deployed, the metal stent is brought up along the same guide wire and deployed. During this step, the DPD serves to trap any emboli that may be dislodged during stent deployment. After stent deployment, the DPD is collapsed and withdrawn into its protective sheath.

In the present method and as shown in FIGS. 11 and 14, the use of a DPD would generally not be necessary and thus can save the time used to deploy the DPD as well as the expense of this equipment.

That is, as the resorbable PS of the subject system has a pore size similar to the pore size of a DPD, that is in the range of about 110-250 microns, the act of deploying the resorbable PS will provide the same emboli capturing capabilities of a DPD insomuch as the resorbable PS is self-expanding. In other words, as the resorbable PS deploys distally to the plaque, the distal end will expand against the intima and progressively be deployed in the proximal direction. Thus, any emboli 902b breaking free from the plaque during deployment will be caught between the PS and the intima as shown in FIGS. 11 and 14. Importantly, during this step, the surgeon should ensure that the PS is deployed sufficiently distal to the plaque that the distal tip of the PS is fully contacts the vessel before the PS is deployed across the plaque. This will generally require that the PS is long enough to be deployed in a straight distal section of the ICA.

This technique by virtue of the PS pore size, which is significantly smaller than a typical metal stent will thus retain any emboli between the PS and the intima. Importantly, in situations where the PS is resorbing over time, the emboli will also be resorbed into the intima and/or dissolved as a result of normal blood thinning regimes.

### EQUIVALENTS

At least the following equivalents and scope are contemplated.

An example location for the unstable plaque 404 is described with respect to FIGS. 4 to 13. However, this location is merely exemplary. An unstable plaque may be located in the CCA 400a or the ICA 400b or a combination thereof. The geometry of the resorbable PS would be readily apparent to the skilled person in view of the discussion provided herein.

FIGS. 4 to 13 contemplate balloon deployment in each of the CCA and the ECA to substantially arrest blood flow at an unstable plaque. It will be appreciated that occlusion of at least any two of the three arteries proximal to the CCA bifurcation could substantially arrest blood flow at the unstable plaque.

If one or more balloons are used to substantially arrest blood flow at an unstable plaque, it will be appreciated that the balloons may be deflated either by manual input by someone operating the BGC or may automatically deflate after a predetermined period of time. In a further embodiment, the distal balloons may be a self deflating detachable balloon that may be detached into the ECA.

### Uses and Kits

In addition to the methods described above, uses of PSs and resorbable PSs and kits are also contemplated. The uses and kits described below encompass at least features described in the methods disclosed above and its equivalents.

A use of a resorbable PS is contemplated. Specifically, the use may be of a resorbable PS to stabilize an unstable plaque in a patient for a therapeutically effective time period at a bifurcation of a CCA into an ICA and an ECA, where the resorbable PS is deployed under substantial arrest of blood flow at the unstable plaque.

A kit for the treatment of an unstable plaque in a patient is also contemplated. The kit may include one or more devices, the one or more devices adapted to substantially arrest blood flow at the unstable plaque adjacent to a bifurcation of a CCA into an ICA and an ECA. The kit may further include or merely comprise at least one resorbable PS adapted to stabilize the unstable plaque for a therapeutically effective time period.

Kits may comprise within individual or separate packing a combination one or more of a first BGC, a second BGC that is deployable through the first GBC, one or more guide wires, one or more microcatheters and one or more PS assemblies having one or more resorbable PSs. The resorbable PSs may be provided with a variety of features that allow a surgeon to select desired functional and structural characteristics for a specific case.

For example, PSs may have combinations of the following functional/structural characteristics including a range of:
diameters appropriate for the vessel;
lengths appropriate for the vessel;
tapers appropriate for the vessel;
compressive stiffnesses appropriate for the design of the deployment system;
pore sizes appropriate to provide distal protection functionality balanced against other design parameters;
filament composition appropriate to provide desired pore and stiffness properties;
filament diameters appropriate to provide desired pore and stiffness properties;
drug coatings appropriate for a patient's treatment protocol; and,
resorb times appropriate for a patient's treatment protocol and other design parameters.

Various PSs may have different combinations of each of the above structures and functionalities.

### Plaque Stabilizer Design

As noted, a PS may have a plurality of features that make it suitable for use in treating unstable plaque. Given the variability in the size and location of plaque being treated adjacent the CCA bifurcation, PSs having different lengths and features may be utilized.

For example, a plaque in the ICA may be 7-9 mm in length and extend into the ICA 0.5-1 mm. The center of the plaque may be 4-6 mm from the bifurcation. Generally, in order to enable the PS to be useful as a DPD, the PS would typically be longer than a PS that is used with a separate DPD.

That is, as shown in FIG. 14, as the PS must contact the intima before it is fully effective as a DPD, and there is a distance between the distal tip of the PS and the distal tip of the deployment catheter before the distal tip of the PS is fully engaged with the intima, the surgeon will typically need to deploy the PS a few mm further in the distal direction to enable this. Hence, in comparison to current stents used at this location, the PS in accordance with the invention will typically be a few mm longer. Moreover, particularly when the procedure is conducted without flow cessation, the initial step of PS deployment should be conducted further in the distal direction to minimize contact with the plaque and the risk of disturbing it. As such, a PS will typically be 30-50 mm long and more specifically 40-42 long.

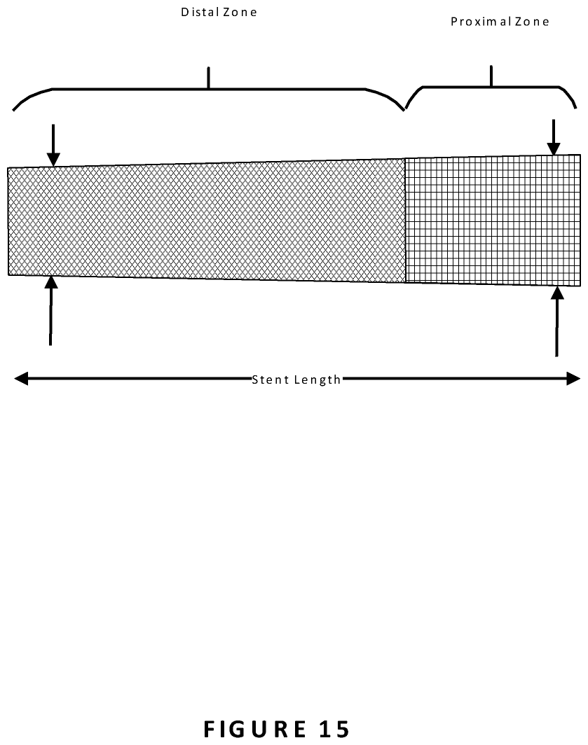

As shown in FIG. 15, the PS may also include different zones having different porosities. That is, as the PS is deployed in the proximal direction, and as described above, it will generally extend into the CCA. As a small pore size PS will generally be restrictive to blood flow, the PS may be provided with a proximal zone having a larger pore size. The proximal zone pore size would typically be larger than 250 microns, whereas the distal pore size would be in the range of 110-250 microns. Generally, in the distal zone, the void space would be greater than 50% and the PS would be a braided cylinder of resorbable filaments having a filament diameter in the range of 30-50 microns, typically 40 microns.

The relative proportions of length of the proximal and distal zones would typically be 20-30% proximal and 70-80% distal as shown in FIG. 15.

The ultimate selection of the length and other features of the PS will be determined by the surgeon having regard to the particular characteristics of the plaque.

It should also be noted that braided metal PSs having the above structural features could be developed and utilized. In particular, these PSs could also be effective as DPDs as described above.

### CONCLUSION

While this invention has been particularly shown and described with references to embodiments thereof, it will be understood by those skilled in the art that various changes in form and details may be made therein without departing from the scope of the invention encompassed by the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A plaque stabilizer (PS) for deployment over an unstable plaque/web/thrombus comprising:
a cylindrical body having a plurality of pore openings in the range of 110-250 microns diameter and a void space of greater than 50% of the cylindrical body, the cylindrical body collapsible within a microcatheter and deployable from the microcatheter for placement over the unstable plaque/web/thrombus and wherein the cylindrical body is self-expanding upon deployment within an artery.

33. The PS as in claim 32 wherein the PS is comprised of a resorbable material having a resorb time of one week or less.

34. The PS as in claim 32 wherein the PS is comprised of a resorbable material having a resorb time of one month or less.

35. The PS as in claim 32 wherein the PS is comprised of a resorbable material having a resorb time of two months or less.

36. The PS as in claim 32 wherein the PS is poly lactic-co-glycolic acid.

37. The PS as in claim 36 wherein the cylindrical body is a weave of poly lactic-co-glycolic acid filaments, the filaments having a diameter in the range of 30-50 microns.

38. The PS as in claim 32 wherein the PS has at least two zones, a first distal zone having pore opening in the range of 110-250 microns diameter and a second proximal zone having pore openings greater than 250 microns.

39. The PS as in claim 32 wherein the cylindrical body has an overall length of 3-5 cm.

40. The PS as in claim 38 wherein the first distal zone is 70-80% of the overall length of the cylindrical body.

41. The PS as in claim 38 wherein the second proximal zone is 20-30% of the overall length of the cylindrical body.

42. The PS as in claim 32 where the PS is metal.

43. A kit for the treatment of an unstable plaque at or adjacent to a bifurcation of a common carotid artery (CCA) to an internal carotid artery (ICA) and external carotid artery (ECA) in a patient, the kit comprising:
at least one guide catheter (GC) configured for placement of the GC proximal to the unstable plaque;
at least one plaque stabilizer deployment device (PSDD) configured for telescopic engagement within the GC and for placement distal to the unstable plaque;
at least one guide wire (GW) configured for telescopic engagement within the MC and for placement distal to the unstable plaque;
at least one PS configured to the PSDD for placement adjacent to the unstable plaque and deployable through from the PSDD.

44. The kit as in claim 43 where the PS is resorbable over a resorb time.

45. The kit as in claim 43 where the GC is at least one balloon guide catheter (BGC) for occluding blood flow through the CCA.

46. The kit as in claim 43 further comprising at least one micro-balloon (MB) for occluding blood flow through the ECA.

47. The kit as in claim 43 where the kit includes at least two resorbable PS assemblies each having a resorbable PS, and where the resorbable PSs have at least one different structural and/or functional property from each other, selected from any one of or a combination of PS diameter, PS length, PS taper, PS compressive stiffness, PS pore size; PS drug coating and PS resorb time.
